(19) United States
(12) Patent Application Publication
(10) Pub. No.: US 2022/0142485 A1
(43) Pub. Date: May 12, 2022

(54) DIAGNOSTIC TOOL BASED HEALTH MANAGEMENT SYSTEM

(71) Applicant: THE REGENTS OF THE UNIVERSITY OF CALIFORNIA, Oakland, CA (US)

(72) Inventors: Samir G. Shreim, Irvine, CA (US); John Weidling, Irvine, CA (US); Mark Keating, Irvine, CA (US); Elliot L. Botvinick, Irvine, CA (US); Sean Michael White, Irvine, CA (US)

(21) Appl. No.: 17/586,137

(22) Filed: Jan. 27, 2022

Related U.S. Application Data
(63) Continuation-in-part of application No. PCT/US20/42054, filed on Jul. 15, 2020.
(60) Provisional application No. 62/880,568, filed on Jul. 30, 2019.

(51) Int. Cl. A61B 5/00; G16H 50/20; G16H 30/40; G16H 40/67; A61B 1/32; A61B 1/06; A61B 1/00; A61B 1/04; A61B 1/227

(52) U.S. Cl. CPC A61B 5/0075; G16H 50/20; G16H 30/40; G16H 40/67; A61B 1/227; A61B 1/0638; A61B 1/00048; A61B 1/042; A61B 1/32

(57) ABSTRACT

A point-of-care system with a diagnostic tool and a base station with a display and in communication with the diagnostic tool is described. Used in diagnosing health conditions for a tissue under analysis, the diagnostic tool includes a handle and a head portion. The head portion includes a speculum and optical spectroscopy (OS) data acquisition components positioned within the head portion. The OS data acquisition components are configure to (i) emit light toward the tissue under analysis, (ii) receive light reflected at least in part from the tissue under analysis based on the emitted light, and (iii) determine reflectance spectra associated with the received light. Either the diagnostic tool or a base station includes analytic components configured to (i) generate diagnostic metrics including characteristics of the reflectance spectra and (ii) compare these characteristics to data associated with characteristics of known reflectance spectra associated healthy and/or unhealthy tissue of patients.

### CROSS-REFERENCE TO RELATED APPLICATIONS

This application is a continuation-in-part and claims benefit of PCT Application No. PCT/US20/42054 filed Jul. 15, 2020, which claims benefit of U.S. Provisional Patent Application 62/880,568 filed on Jul. 30, 2019, the entire contents of which are incorporated by reference herein.

### FIELD

Embodiment of the disclosure relates to the field of health management. More particularly, one embodiment of the disclosure relates to a health management system for retention and conveyance of diagnostic, optical spectroscopy (OS) measurements based on analyses using a diagnostic tool for assessing the health condition of a patient.

### GENERAL BACKGROUND

Otitis Media (OM), or middle ear inflammation is the second most common cause for pediatrician office visits after the common cold. OM accounts for over 20 million office visits per year in the United States alone, with 75% of these being children under 3 years old. Acute Otitis Media (AOM) is a common, usually painful, type of OM that results from an abrupt onset infection for which antibiotics are usually prescribed. Given that AOM inflicts young children, especially children under three years of age, AOM is frequently misdiagnosed.

Poor diagnostic accuracy of AOM results from several factors. Initially, in evaluating a patient for AOM, a physical examination is conducted using an otoscope to visually inspect the middle ear of a feverish child having an earache. In many cases, a clinician may need to remove cerumen (ear wax) from the patient's ear canal to visually inspect the middle ear using a conventional otoscope, which may be extremely painful for the patient. Given the young age of most patients being evaluated for AOM (and the pain cause through wax removal), current AOM analyses through use of conventional otoscopes are grossly inaccurate as patients tend to move and “act out” during examination. Also, AOM analyses are singularly designed to diagnose and address the patient's current ear infliction, without any consideration on a wealth of heuristics (e.g., diagnosis results of similarly situated patients, geographic trends such as AOM detection is specific regions, etc.) that may alter the diagnosis and improve overall diagnosis accuracy by reducing the number of false positives and/or false negatives.

More specifically, while visual inspection for OM may assist in determining the presence of AOM, this inspection does not augment the level of care that could be given to the patient during OM analyses. Recently, efforts have been made to utilize reflected light in accordance with optical spectroscopy (OS) technologies to assess the health of the middle ear. However, current health services have failed to develop a suitable platform for the collection, retention and utilization of heuristics associated with optical spectroscopy (OS)-based diagnostics. These heuristics may include metrics that identify healthy or unhealthy (e.g., AOM, OM, etc.) tissue. From the heuristics, clinicians and home users would be able to better assess the presence of an ailment that requires antibiotics or an office visit from symptoms that may have erroneously lead to an office visit that wastes the patient's and any family member's time and/or unnecessary prescriptions that waste financial resources.

### BRIEF DESCRIPTION OF THE DRAWINGS

Embodiments of the invention are illustrated by way of example and not by way of limitation in the figures of the accompanying drawings, in which like references indicate similar elements and in which:

FIG. 1 is a first exemplary embodiment of a health management system including a Point-of-Care (POC) system.

FIGS. 2A-2B are illustrative embodiments of the POC system of FIG. 1 with wired connectivity between a base station and a diagnostic tool implemented as part of the POC system.

FIG. 3A is an illustrative embodiment of the POC system of FIG. 1 with wireless connectivity between the diagnostic tool and the base station.

FIG. 3B is an illustrative embodiment of the POC system of FIG. 1 with wireless connectivity between the diagnostic tool and a cellular telephone (smartphone).

FIG. 4A is a first illustrative embodiment of a fragmentary cross-sectional perspective of the diagnostic tool of FIGS. 1-3B for diagnosing health conditions, such as ear conditions including different types of Otitis Media (OM).

FIG. 4B is a second illustrative embodiment of a fragmentary cross-sectional perspective of the diagnostic tool of FIGS. 1-3B for diagnosing health conditions, such as different types of Otitis Media (OM).

FIG. 5A is a first illustrative embodiment of a camera-based diagnostic tool.

FIGS. 5B-5C is a second illustrative embodiment of a camera-based diagnostic tool.

FIG. 5D is a third illustrative embodiment of a camera-based diagnostic tool.

FIG. 5E is a fourth illustrative embodiment of the diagnostic tool with visualization logic and without cameras deployed within a head portion of the diagnostic tool.

FIG. 6 is a second exemplary embodiment of the health management system.

FIG. 7 is an exemplary embodiment of a logical architecture of the diagnostic tool of FIG. 1.

FIG. 8A is a first exemplary embodiment of the operations of the diagnostic tool operating within the health management system of FIG. 6.

FIG. 8B is a second exemplary embodiment of the operations of the diagnostic tool operating within the health management system of FIG. 6

### DETAILED DESCRIPTION

One embodiment of the disclosure is directed to a health management system that features a point-of-care (POC) system, which may be communicatively coupled to one or more networks that provide, either directly or indirectly, storage of examination results and/or processing of optical spectroscopy-based (OS) data to generate the examination results. Representing the condition of tissue under diagnosis (e.g., middle ear, throat, intestine, etc.), these examination results may include a collection of diagnostic metrics, which are calculated based on captured light emitted from and returned to a diagnostic tool, namely part of the POC system, after interaction (e.g., scatter and/or absorption) with matter within a body cavity in which the tissue resides. The “diagnostic metrics” represent data associated with characteristics (e.g., a parameter or combination of parameters such as shape, magnitude, etc.) of the OS data that is based on the captured emitted light, referred to as “reflectance spectra.” These characteristics are useful in detecting a health condition of tissue under examination. For example, the characteristics may represent hemoglobin levels of a patient, which may be used in determining the overall health of that patient.

Besides the diagnostic metrics, according to one embodiment of the disclosure, the POC system may be configured to process and subsequently store image data from a set (one or more) of imaging components (e.g., one or more cameras, etc.) installed within the diagnostic tool as described below. Each of the imaging component(s) may capture one or more images, which may include color images or black/white images, where different wavelengths of light are used to illuminate the body cavity. These different wavelengths may be selected so as to cause greater imaging contrasts to occur for different conditions of the ear to which a clinician might be interested. For example, illumination with certain wavelengths of light may be used to achieve greater imaging contrasts for a first type of ailment (e.g., AOM) to better identify the presence of certain compositions of matter within the body cavity, such as water or hemoglobin for example. Different wavelengths may be used to achieve greater imaging contrasts for a second type of ailment (e.g., strep throat).

The deployment of imaging components provides a number of advantages. For example, the use of imaging components provides enhanced accuracy in diagnosing of ailments (e.g., AOM) by relying on the examination results for a preliminary diagnosis and confirming the diagnosis by analysis of the images (e.g., review of imaging contrasts). Another advantage is directed to the deployment of imaging components used for optical tympanometry (analysis of the middle ear), where the imaging components may be used to stereoscopically interrogate the shape of the eardrum, and then even provide, for example, a cross-sectional view of the degree of distention or retraction based on depth computations from the imaging components. Stated differently, the imaging components, when deployed, may capture information for use in detecting a bulging or retracting tympanic membrane.

According to one embodiment, the POC system may be implemented where the components that process the OS data (hereinafter, “OS analytic components”) may be located within the diagnostic tool along with the components that collect the reflectance spectra for conversion to the OS data (hereinafter, “OS data acquisition components”). Herein, the OS analytic components and the OS data acquisition components may be generally referred to as “OS components.” It is contemplated that, in accordance with an alternative embodiment, the acquisition and processing of the OS data may be conducted at different devices within the POC system. More specifically, the OS data acquisition components may be implemented within the diagnostic tool while the OS analytic components may be located external to the diagnostic tool (e.g., within a base station located in close proximity to or within another electronic device remotely located from the diagnostic tool). These different POC system implementations (e.g., localized, centralized, etc.) are described below.

According to another embodiment, the POC system may be implemented to enable clinicians and/or patients to input qualitative metrics to enhance diagnostic performance. In one example, data related to a patient's age, sex, allergy status, or the like may be input by the clinician and used by the POC system as part of a broader diagnostic in which the input qualitative metrics may be used to provide context to one or more images generated by the set of imaging components. In some cases, it may be desirable to use the POC system as a predictive tool, where the OS data is used alone or in conjunction with qualitative metrics to prospectively predict specific health indicators. In one example these health indicators may be related to otitis media. In another example these health indicators may be related to otitis externa. Alternatively, the POC system (or certain functionality of the POC system) may be used to determine ear health for determining candidacy of a hearing aid or for use in the physical development of ear tubes or another hearing assistance devices or placement of a particular type or types of additive (e.g., coating with certain medicinal agents) directed to mitigate a particular condition detected by the POC system.

I. General Summary

As described herein, the POC system includes one or more diagnostic tools (referred to in singular form as “diagnostic tool” for simplicity sake) and a base station. According to one embodiment of the disclosure, the diagnostic tool is configured with OS data acquisition components to obtain the OS data. However, depending on the deployment selected for the health management system as described below, the OS analytic components responsible for processing of the OS data, obtained via the diagnostic tool, may be deployed within (a) the diagnostic tool, (b) the base station, (c) both the base station and the diagnostic tool, and/or (d) one or more electronic devices in communication with the diagnostic tool and/or the base station. These OS analytic components may include physical components (e.g., hardware processor, memory, etc.) and/or virtual components (e.g., virtual machines, software instances with different functionality, services such as compute and/or storage services within a private or public cloud network such as Amazon Web Services®, Microsoft® Azure®, or the like).

Herein, in accordance with a first embodiment of the disclosure, the POC system may be deployed as part of a local network for a clinician, where the diagnostic tool is communicatively coupled to the base station. For this embodiment, the diagnostic tool may be configured with OS data acquisition components that, when in operation, collect the OS data associated with tissue within a body cavity under examination by at least (i) emitting light from multiple spectral illumination light sources having different wavelengths, (ii) capturing the reflectance spectra for the emitted light, and (iii) converting the reflectance spectra into an acceptable format (OS data) for processing. The capturing of the reflectance spectra may be conducted during optical tympanometry. For this embodiment, the diagnostic tool may include OS analytic components to generate the diagnostic metrics from the OS data. The diagnostic metrics collectively formulate the examination results, which are downloaded to one or more selected resources (e.g., base station, server within a local or cloud network, etc.) being part of the health management system.

For a second embodiment of the disclosure, the POC system may be deployed as part of a centralized diagnostic system, where the above-described reflectance spectra is captured, converted into the OS data for processing by a diagnostic tool, but the OS data is provided to components within the base station. The base station processes the received OS data to determine the health condition of tissue under examination, such as the middle ear for example. The base station is accessible over a public network (e.g., Internet) and configured to receive and process OS data from the diagnostic tool or multiple (two or more) diagnostic tools. Herein, the base station associated with a centralized diagnostic system may be maintained by a governmental entity or one or more health service providers (e.g., hospital(s), clinic(s), clinician's office(s), a pharmacy or network of pharmacies), and/or an insurance company or a network of insurance companies, etc.).

According to one embodiment of the disclosure, within the diagnostic tool, certain OS data acquisition components collectively operate to illuminate matter accessible via an ear canal (e.g., portions of the ear canal, tympanic membrane, middle ear cavity, cerumen, air and/or fluid in the middle ear cavity, etc.) as well as detect and collect light returning to the diagnostic tool after interaction (e.g., scatter and/or absorption) with matter within the ear cancel. This captured light (e.g., reflectance spectra) is converted into OS data. The OS data may be provided to the OS analytic components, situated and operate within the diagnostic tool (e.g., head and/or handle of an otoscope), which analyzes the OS data to generate diagnostic metrics. The diagnostic metrics may include characteristics of the reflectance spectra, as described above.

For instance, according to one embodiment of the disclosure, the assessed characteristics may be compared to (i) data associated with reflectance spectra for healthy patients (e.g., patients with healthy tissue in ears, throats, intestines, etc.) and/or (ii) data associated with reflectance spectra for tissue of patients exhibiting health conditions, including ailments such as different types of Otitis Media (OM)—Acute Otitis Media (AOM) or Otitis Media with effusion (OME) for example. As an illustrative example, the accessed characteristics may be used to identify a downward trending of hemoglobin level, perhaps over a prescribed period of time and detection of OM, may indicate that an ear infection is resolving and continued monitoring is a preferred treatment in lieu of antibiotics. In a second example, the assessed characteristic may be used to monitor hemoglobin levels for the patient over a longer prescribed period of time that suggests nutritional assistance or counsel. As yet another illustrative example, the accessed characteristics may be used to triage high risk patients needing ear tubes based on a prolonged OME condition or a determined pattern of symptoms which, based on heuristic data, have resulted in language development delays due to hearing loss.

More specifically, according to one embodiment of the disclosure, the OS components deployed within the diagnostic tool may utilize returned (e.g., reflected) light to detect tissue properties. Light incident on turbid media, such as the eardrum or infected middle ear for example, is both absorbed and scattered, namely matter residing in the middle ear absorbs and scatters light in a particular way that may be assessed spectrally. This creates a diffuse and unique reflectance spectra (e.g., reflective and/or chromatic spectra), which may be subsequently used as a spectral reference profile. The diffuse nature of optical spectroscopy enables measurements in situations that preclude optical evaluation, such as an ear canal occluded by wax. Provided the path length is long enough that transmitted or back-reflected light is diffused, the Beer-Lambert Law provides the diagnostic tool with an accurate approximation to quantitatively determine the concentration of tissue chromophores:

$$I ( λ ) = {I}_{0} ( λ ) {10}^{- {μ}_{s}^{′} ( λ ) l - ( {\sum }_{i} {μ}_{a , i} ( λ ) ) l} \qquad Equation ( 1 )$$

where “I(λ)” is the measured light intensity as a function of wavelength λ, “I₀(λ)” is the incident intensity, “μ_{a,i}(λ)” is the i^{th} chromophore's absorption coefficient, “μ's(λ)” is the scattering coefficient and “1” is the optical path length. Herein, the provided “μ_{a,i}(λ)” is known (e.g., oxyhemoglobin and/or deoxyhemoglobin) and scattering and path length can be estimated, inversion of Equation (1) provides accurate, quantitative information on tissue properties.

Herein, the diagnostic tool may be broadly defined as any hand-held measurement device that analyzes the health of an individual (e.g., tissue within a body cavity such as the middle ear, throat, intestine, etc.) through analysis of diagnostic metrics (e.g., assessed characteristics). The diagnostic metrics are determined based on the OS data computed by reflectance spectra, for comparison with (i) diagnostic metrics associated with prior measured reflectance spectra for the patient, (ii) diagnostic metrics associated with prior measured reflectance spectra for other patients within similar symptoms in which certain treatments have been effective or ineffective, (iii) diagnostic metrics associated with prior measured reflectance spectra for healthy tissue, and/or (iv) diagnostic metrics associated with prior measured reflectance spectra for tissue with Otitis Media (OM) or other health conditions. In some embodiments, the OS data may be combined with qualitative metrics about the patient to enhance diagnostic performance. Some examples of the qualitative metrics include, but are not limited to, age, sex, allergy status, otitis media history, or other medical history data.

More specifically, the diagnostic tool may be deployed as an otoscope, endoscope, or another type of optical-based clinical device that at least include the OS data acquisition components oriented in accordance with a selected geometry to emit light and collect reflected and/or refracted light for analysis and detection of types of matter resident in a body cavity (e.g., ear canal). For instance, the OS data acquisition components may include one or more spectral illumination light sources, such as a multiple light emitting diode (multi-LED) chip, each LED configured to illuminate tissue within the body cavity with light of a prescribed range of wavelengths (e.g., each wavelength range may span approximately 50 nanometers (nm) or more and entire wavelength spectrum may vary from 500 nm to 1450 nm or more). The reflectance spectra may be captured by selectively positioned light detection elements (e.g., photodetectors, etc.) within the diagnostic tool such as described in a Patent Cooperative Treaty (PCT) Patent Application No. PCT/US19/33393 entitled “Light-Emitting Diode Based Diffuse Optical Spectroscopy Tool For Assessing the Ear,” filed on 21May 2019, the entire contents of which are incorporated by reference herein. As optional components, the OS data acquisition components may further include one or more imaging components (e.g., one or more cameras).

As an illustrative example, the spectral illumination light sources, also referred to as a “spectral light sources,” may include, but is not limited or restricted to one or more LEDs such as the multi-LED chip described above. Additionally, or in the alternative, the spectral light sources may include one or more laser diodes, vertical cavity surface emitting lasers (VCSELs), and/or broadband (e.g., incandescent, fluorescent, etc.) light sources. Each light detection element may include, but is not limited or restricted to a photodetector such as a photodiode (e.g., silicon and InGaAs), spectrometer on a chip (e.g. CCD and CMOS based spectrometers on a chip), or the like.

In addition to OS data acquisition components, the diagnostic tool may include interface components (e.g., scan button, display screen with perhaps touch sensitivity or selection buttons located on the bezel, etc.), administrative components (e.g., scan management logic operating as one or more counters, smart card adapter to receive and access data within an installed smart card, etc.), and/or communication components. Herein, the communication components may provide wireless connectivity to one or more networks or may provide a physical connector for coupling to the base station, such as a docketing station or cellular telephone for example, for use in transferring OS data (when OS analytic components are remoted located from the POC system) or diagnostic metrics, perhaps including one or more images captured by the OS data acquisition components, to a network services that conducts an analysis of the OS data.

Additionally, as optional components, the diagnostic tool may include temperature measuring components, which may be used to identify a temperature of the patient as well as trending temperature values in determining the overall health of the patient based on patient heuristics and potential actions to be undertaken in patient treatment.

As yet another embodiment, as described briefly above, the diagnostic tool may include the imaging components, which may include image capturing logic and/or image rendering logic. More specifically, the image capturing logic is configured to capture an optical image or a series of optical images (e.g., one or more images in temporal sequence, video, etc.). The image rendering logic is configured to convert the image or the series of images into a prescribed format for display on a display screen and/or transfer, using one or more communication components, from the image rendering logic within the POC system to the base station and/or prescribed network.

According to one embodiment of the disclosure, when implemented as an otoscope, the diagnostic tool may be configured to also permit visual investigation of an ear canal along with automated OS-based examination of the ear canal. More specifically, the visualization optics may be installed within the diagnostic tool to enable visual inspection of tissue within a body cavity. The visual inspection may be conducted, concurrently (e.g., at least partially overlapping in time) or in series, with automated OS-based examination of the collected OS data to improve diagnostic accuracy as well as augment otoscope functionality.

As described above, in lieu of deploying the POC system entirely within the local network, according to another embodiment of the disclosure, the POC system may be deployed as part of a centralized diagnostic system accessible over a public network (e.g., Internet) and maintained by a governmental entity or one or more health service providers. For this embodiment, interacting with the base station of the POC system, the diagnostic tool includes the OS data acquisition components while the base station includes the OS analytic components, which receive the OS data and generate diagnostics metrics for subsequent evaluation remotely by a clinician. Herein, the diagnostic tool may further include image capturing components to capture an optical image or a series of optical images (e.g., video) while the base station may include image rendering logic to convert the optical image(s) into an image or the series of images for display on a display screen being part of the base station, a diagnostic tool, or remotely located therefrom.

In the following description, numerous specific details are set forth. However, it is understood that embodiments of the disclosure may be practiced without these specific details. For instance, besides the middle ear as described herein, the diagnostic tool may be configured for use with assessing the health condition of other body parts including, but not limited to a throat, skin, body cavities accessed and/or imaged by endoscopy, or those exposed during surgery. In other instances, well-known structures and techniques have not been shown in detail in order not to obscure the understanding of this description. Those of ordinary skill in the art, with the included descriptions, will be able to implement appropriate functionality without undue experimentation.

References in the specification to “one embodiment” or “an embodiment,” may indicate that the embodiment described may include a particular feature, structure, or element, but every embodiment may not necessarily include that particular feature, structure, or element. Moreover, such phrases are not necessarily referring to the same embodiment. Further, when a particular feature, structure, or element is described in connection with an embodiment, it is submitted that such feature, structure, or element may be deployed in connection with other embodiments whether or not explicitly described.

In the following description, certain terminology is used to describe aspects of the invention. In certain situations, the terms “component” and “logic” are representative of hardware, firmware, and/or software that is configured to perform one or more functions. As hardware, the component (or logic) may include circuitry having data processing and/or storage functionality. Examples of such circuitry may include, but are not limited or restricted to a processor, a programmable gate array, a microcontroller, an application specific integrated circuit, wireless receiver, transmitter and/or transceiver circuitry, semiconductor memory, or any collection of combinatorial circuitry.

Alternatively, or in combination with the hardware circuitry described above, the component (or logic) may be software in the form of one or more software modules, which may be configured to operate as its counterpart circuitry. The software modules may include an executable application, a daemon application, an application programming interface (API), a routine or subroutine, a function, a procedure, a shared library/dynamic load library, an applet, a servlet, source code, or even one or more instructions. The software module(s) may be stored in any type of a suitable non-transitory storage medium, or transitory storage medium (e.g., electrical, optical, acoustical or other form of propagated signals such as carrier waves, infrared signals, or digital signals). Examples of non-transitory storage medium may include, but are not limited or restricted to a programmable circuit; a semiconductor memory; non-persistent storage such as volatile memory (e.g., any type of random access memory “RAM”); persistent storage such as non-volatile memory (e.g., read-only memory “ROM”, power-backed backed RAM, flash memory, phase-change memory, etc.), a solid-state drive, hard disk drive, an optical disc drive, or a portable memory device. As firmware, the component (or logic) may be stored in persistent storage.

The term “message” generally refers to signaling (wired or wireless) as either information placed in a prescribed format and transmitted in accordance with a suitable communication protocol or information made accessible through a logical data structure such as an Application Programming Interface (API). Hence, each message may be in an analog form or a digital form such as one or more packets, frames, or any other series of bits having the prescribed, structured format.

In certain instances, the terms “compare,” comparing,” “comparison,” or other tenses thereof generally mean determining if a match (e.g., identical or a prescribed level of correlation) is achieved between different data.

Lastly, the phrases “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A” alone; “B” alone; “C” alone; “A and B”; “A and C”; “B and C”, or “A and B and C.” An exception to this definition will occur only when a combination of elements, functions, steps or acts are in some way inherently mutually exclusive.

Any feature or combination of features described herein are included within the scope of the invention provided that the features included in any such combination are not mutually inconsistent as will be apparent from the context, this specification, and the knowledge of one of ordinary skill in the art. Additional advantages and aspects of the present invention are apparent in the following detailed description and claims. Stated differently, this invention is susceptible to embodiments of many different forms, and thus, it is intended that the disclosure be considered as an example of the principles of the invention and not intended to limit the invention to the specific embodiments shown and described.

II. Health Management System Embodiments

A. General Architecture

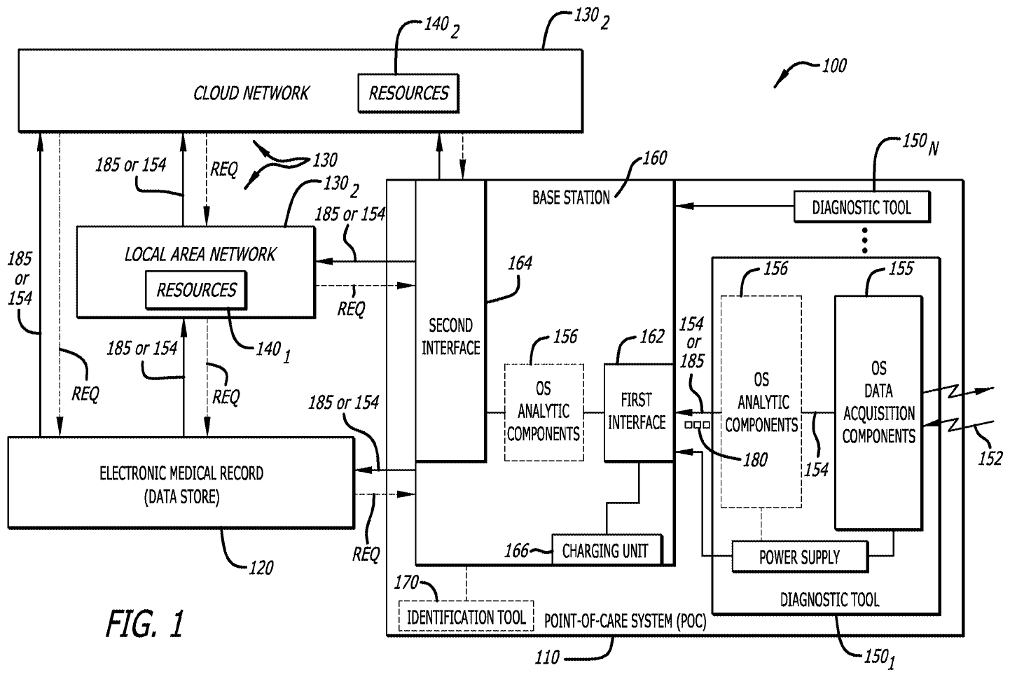

Referring now to FIG. 1, a first exemplary embodiment of a health management system 100 is shown. The health management system 100 comprises a point-of-care (POC) system 110, which is communicatively coupled to a data store 120 for electronic medical records 120 and/or one or more networks 130 (hereinafter, “network(s)”), such as a local area network 1301 or a cloud network 1302. For this embodiment of the disclosure, each of the network(s) 1301/1302 may be configured with resources 1401/1402, which are configured to (i) process OS data collected by the POC system 110, (ii) store diagnostic metrics forming an examination result produced by the POC system 110 during a patient examination, or (iii) a combination of both OS data processing to generate the diagnostic metrics and storage of the diagnostic metrics forming the examination results.

The POC system 110 includes one or more diagnostic tools 1501-150N (N≥1) (generally referred to as “diagnostic tool 150”), a base station 160 and an optional identification tool 170. Herein, the diagnostic tool 150 is configured with one or more components 155 that, when in operation, emits beams of light of different wavelengths to illuminate matter within a body cavity being examined and collect the reflectance spectra 152 for conversion to the OS data 154 (hereinafter, “OS data acquisition components” 155). In particular, a portion of the diagnostic tool 150 is inserted into the body cavity (not shown), such as a portions of the ear canal, and thereafter, OS data acquisition components 155 collect reflectance spectra 152 as light returns to the diagnostic tool 150 after interaction (e.g., scatter and/or absorption) with the illuminated matter.

The diagnostic tool 150 may be further configured with OS analytic components 156 (as shown) that, when in operation, convert the OS data 154 into diagnostic metrics 180. The diagnostic metrics 180 may include characteristics associated with the OS data 154, where these characteristics identify a health condition of the tissue under examination (e.g., middle ear of the patient). These characteristics may represent hemoglobin levels or other biological or chemical compositions.

According to another embodiment of the disclosure, in lieu of being implemented within the diagnostic tool 150, the OS analytic components 156 may be implemented within the base station 160. As below, the OS analytic components 156 at least partially processes the OS data 154 to generate the diagnostic metrics 180, which collectively form examination results 185. In particular, the processing of the OS data 154 may be conducted by (a) OS analytic components 156 implemented within the diagnostic tool 150, (b) OS analytic components 156 implemented within the base station 160, (c) OS analytic components 156 implemented within both the diagnostic tool 150 and the base station 160, or (d) OS analytic components 156 implemented within one or more network devices in communication with the diagnostic tool 150 and/or the base station 160.

Where the processing of the OS data 154 is conducted by resources 140₁ and/or 140₂ deployed within network 130₁ and/or 130₂, these resources 140₁ and/or 140₂ may include the OS analytic components 156. For this deployment, the OS analytic components 156 may be implemented as virtual components operating as compute services within a cloud network 130₂ (e.g., EC2 compute services within Amazon Web Services®). Additionally, or in the alternative, the OS analytic components 156 may be implemented as physical components (e.g., hardware processors, etc.).

Stated differently, in accordance with the first embodiment of the disclosure, the POC system 110 may be deployed to be communicatively coupled to (or part of) a network for a clinician, where the diagnostic tool 150 is communicatively coupled to the base station 160. For this embodiment, the diagnostic tool 150 may be configured with at least the OS data acquisition components 155 to generate the OS data 154 from the reflectance spectra 152 associated with the tissue within a body cavity under examination. The OS data 154 is produced from the reflectance spectra 152, which is recovered during an OS-based examination such as optical tympanometry. Thereafter, the diagnostic metrics 180 may be generated based on the OS data 154. For this embodiment, the diagnostic tool 150 may include the OS analytic components 156 to generate the diagnostic metrics 180. The diagnostic metrics 180 are collected to formulate the examination results 185, which are downloaded to base station 160 as shown.

For a second embodiment of the disclosure, the POC system 110 may be deployed as part of a centralized diagnostic system, where the OS data 154 is captured by the diagnostic tool 150 and provided for processing to OS analytic components 156 operating within the base station 160. Herein, the base station 160 performs the diagnostics (e.g., processing) of the received OS data 154 to determine a health condition of the tissue under examination such as the middle ear. The base station 160 may be located proximate to the diagnostic tool 150 or may be remotely located and accessible over a public network (e.g., Internet) to receive the OS data 154. The base station 160 may be maintained by a governmental entity or one or more health service providers.

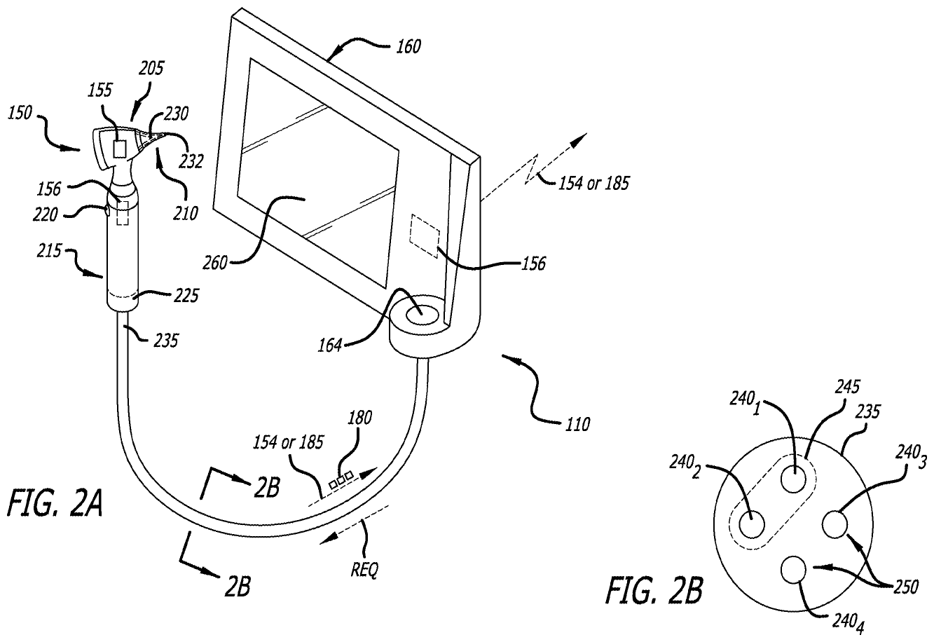

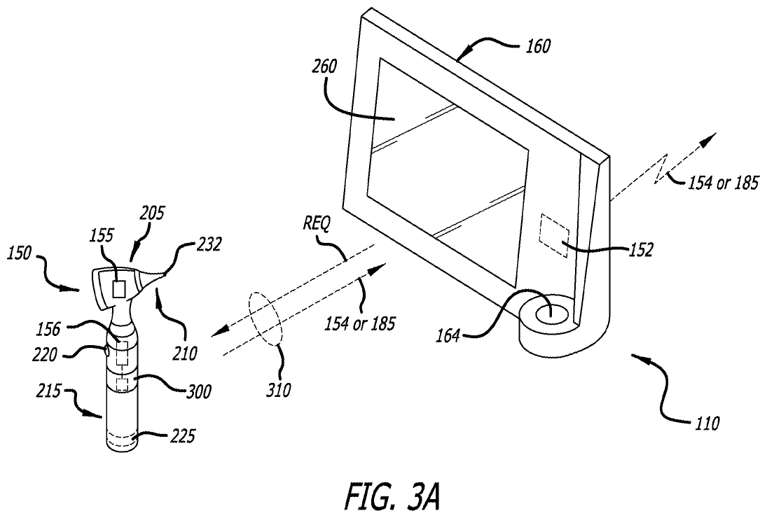

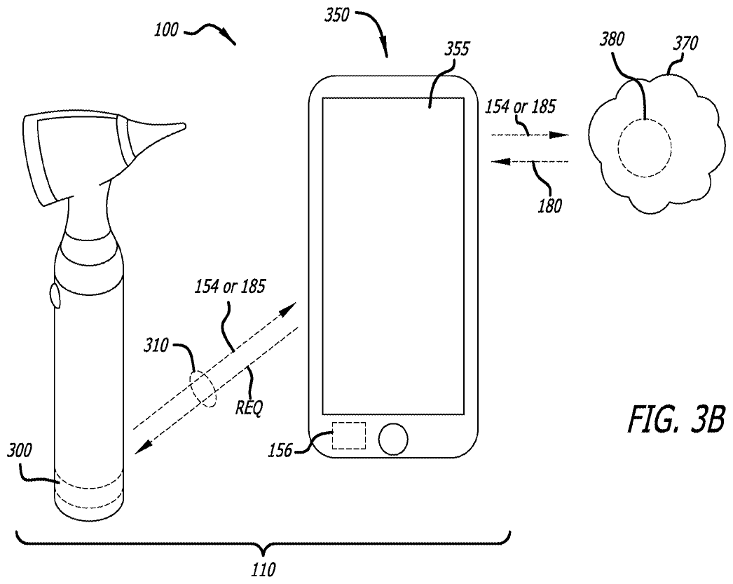

Referring still to FIG. 1, the base station 160 may include at least first interface 162 to support communications with the diagnostic tool 150. Examples of the first interface 162 may include, but is not limited or restricted to, a wireless communication unit (establishing and maintaining wireless communications with the diagnostic tool 150 (see FIGS. 3A-3B) or a wired communication unit such as a physical connector for direct connection (or indirect connection via a wired interconnect) with the diagnostic tool 150 (see FIGS. 2A-2B). Similarly, the base station 160 may include a second interface 164 for use in establishing and maintaining communications between the base station 160 and the network resources 140₁-140₂. Where the first interface 162 is a physical connector, the base station 160 may further couple and provide power from its charging unit 166 to a rechargeable power supply 158 located within the diagnostic tool 150.

As shown, the base station 160 may include OS analytic components 156 to conduct processing of the received OS data 154 to generate the diagnostic metrics 180 as described above. However, it is contemplated that, where the OS analytic components 156 are implemented within the diagnostic tool 150, the base station may operate as a device for uploading the collection of diagnostic metrics, namely the examination results 185, to secure storage. In particular, the base station 160 may be implemented as a docking station, with an Ethernet jack or cable modem connector for connectivity to the local area network 130₁ and/or cloud network 130₂. The base station 160 may include the charging unit 166 to charge the diagnostic tool 150, especially where the first interface 162 provides a mechanical connection with the diagnostic tool 150. Alternatively, the base station 160 may be installed on a recess formed into a wall for placement of the diagnostic tool 150 therein, for collection of the diagnostic metrics 180, local storage of the examination results 185 (and corresponding meta information), and uploading of the examination results 185 (and corresponding meta information) into an electronic file history for the patient as maintained by the electronic medical record 120. The examination results 185 may be stored in the patient's electronic medical record and accessible via networks 130₁/130₂ or the OS data 154 may be stored therein and accessible via networks 130₁/130₂ via request (REQ) messages when “push” communications are deployed.

It is contemplated that, upon or prior to establishing communications that control access to the electronic medical record 120, the patient associated with the examination results 185 may be identified by the identification tool 170. For example, the identification tool 170 may be a scanner that identifies the patient upon scanning a displayable object (e.g., barcode, QR code, etc.) associated with the patient's file, conducting a biometric operation on the patient (e.g., retinal scan, fingerprint scan, facial scan, etc.), or entering data to identify the patient. Moreover, the identification tool 170 may extract an identifier for the patient to be included as part of the examination results 185 by the diagnostic tool 150 or the base station 160.

B. Wired Point-of-Care (POC) System

Referring now to FIGS. 2A-2B, an illustrative embodiment of the POC system 110 with wired connectivity between the base station 160 and the diagnostic tool 150 is shown. As shown in FIG. 2A, the diagnostic tool 150 is a non-invasive otoscope for diagnosing health conditions, such as ear conditions including different types of Otitis Media (OM). The diagnostic tool 150 includes a head portion 205 that includes a removable (and replaceable) speculum 210 and a handle 215 that includes a measure button 220 and a wired interface 225. In particular, the diagnostic tool 150 has been modified to include at least OS data acquisition components 155, which are configured to emit light into a portion of a body cavity (e.g., ear canal) via the speculum 210 after insertion of the speculum 210 into the body cavity and depression of the measure button 220. Additionally, the OS data acquisition components 155 are configured to receive light returning from the body cavity and at least measure the reflectance intensity from a spectra of the returned light (reflectance spectra).

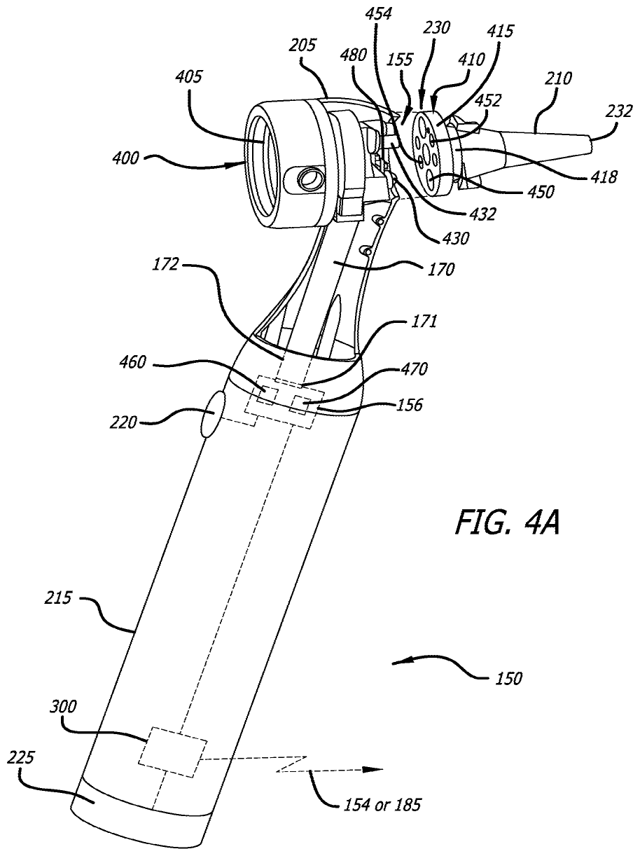

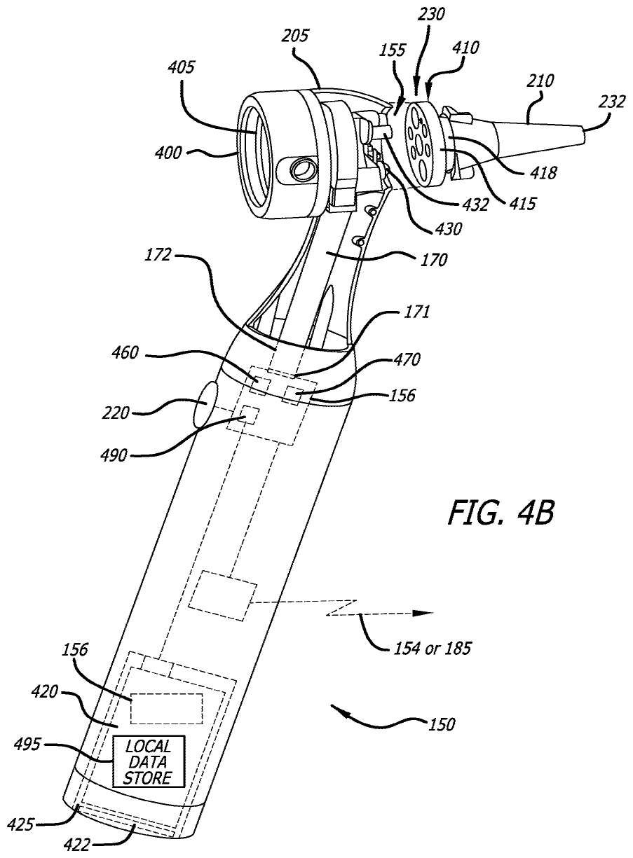

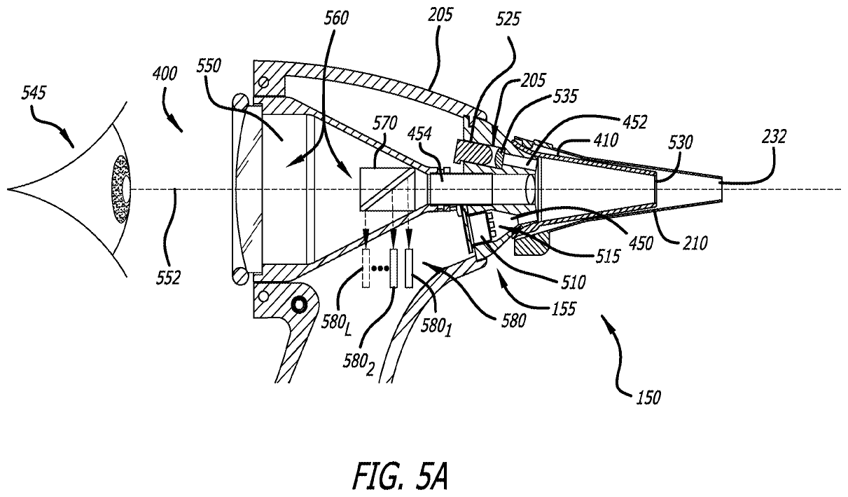

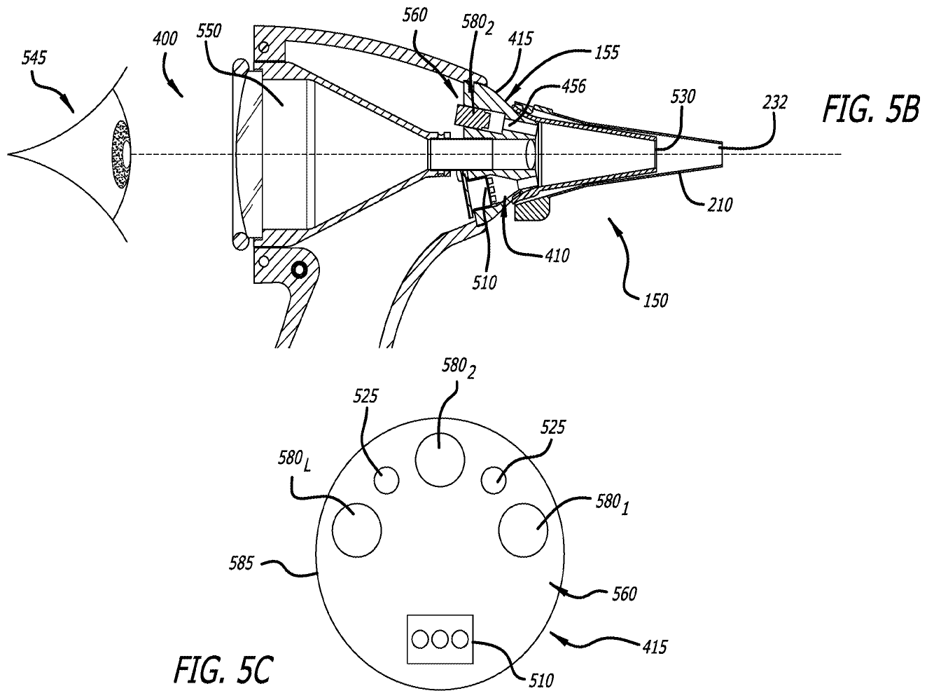

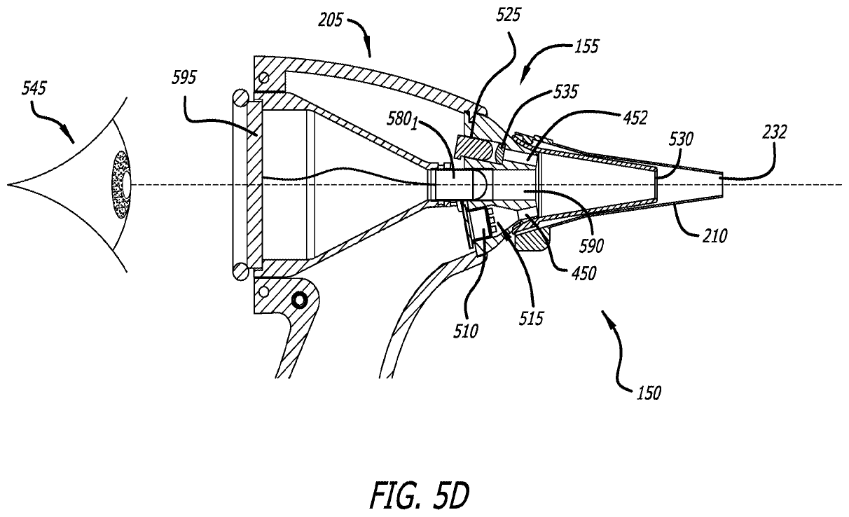

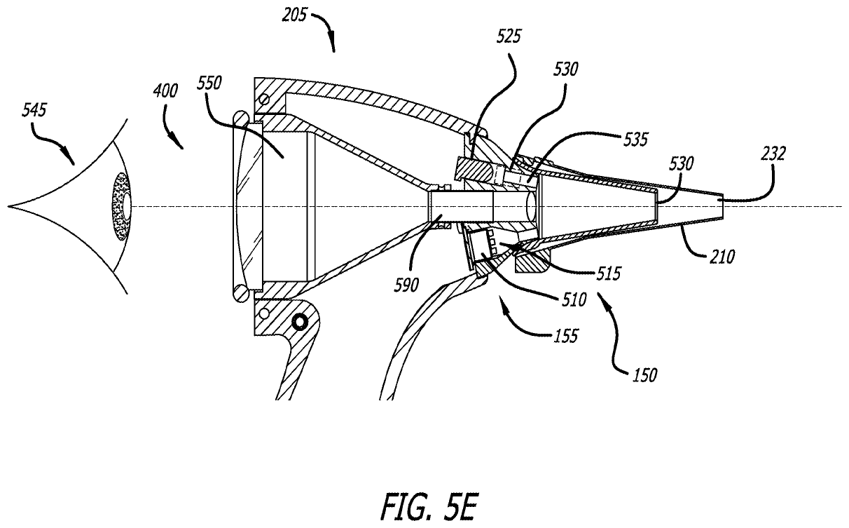

As an optional feature, the head portion 205 may include conduits 230 (e.g., channels, tubes, etc.) directed toward an opening 232 within the speculum 210. The conduits 230 are sized to maintain one or more spectral illumination light sources and one or more light detection elements (see FIGS. 4A-4B). Also, as an optional feature, the conduits 230 may include imaging components (e.g., cameras) to capture images of the tympanic membrane and other areas of the middle ear. An illustrative example of a camera-based diagnostic tool 150 is shown in detail within FIGS. 5A-5D and described below.

According to one embodiment of the disclosure, the OS data acquisition components 155 are further configured to measure the reflectance spectra by converting the reflectance spectra into an acceptable format (OS data), which undergoes one or more analyses by the OS analytic components 156 to ascertain characteristics of the reflectance spectra (e.g., metrics determined through analytics of OS data such as shape and/or magnitude of the reflectance spectra) for use in detecting a health condition of the middle ear. These characteristics constitute the diagnostic metrics 180, which may represent hemoglobin levels of the patient along with the presence of water and/or bacteria in the middle ear. In fact, the reflected light incident on turbid media, which is both absorbed and scattered, creates a diffuse and uniquely reflective (and chromatic) spectra, which may be subsequently used as a spectral reference profile. Hence, multiple profiles, formed by patient testing or “trained” through expected findings may be considered in generating diagnostic metrics 180.

For this embodiment, the examination results 185, as a collection of diagnostic metrics 180, is provided to the base station 160 through the wired interface 225 and cable 235. The results 185 may be uploaded automatically or in response to a request (REQ) message. As part of the examination results 185, the diagnostic tool 150 may include images captured by imaging components deployed within the tool 150, as described below.

As shown in FIG. 2B, an exemplary cross-sectional view of the cable 235 is shown. The cable 235 may be a multi-core cable, namely a cable that includes multiple interconnects 240₁-240_{M} (M≥2) and corresponding connectors (not shown) to facilitate communications of information associated with the examination results 185 or OS data 154 produced from the light associated with different wavelength ranges as captured by one or more photodetectors (not shown). Each photodetector is responsible for capturing a specific light wavelength range and is implemented within the head portion 205 of the diagnostic tool 150.

Furthermore, one or more subsets of the cores (interconnects) 240₁-240_{M} may be shielded from other cores to mitigate noise interference on data associated with the information propagating through the cores 240₁-240_{M}. For instance, as shown in FIG. 2B, a first subset 245 of the cores 240₁-2402 conveying the data associated with the diagnostic metrics 180 may be shielded from a second subset 250 of the cores 2403-2404 (e.g., core 2403 conveying image data captured by a camera maintained by the diagnostic tool 150; core 2404 supplying current/voltage). Additionally, or in the alternative, the entire cable 235 may be shielded to mitigate interference with data associated with the examination results 185 being downloaded to the base station 160.

Referring back to FIG. 2A, the base station 160 may receive the examination results 185, which includes the diagnostic metrics 180, an identifier of the patient (i.e., portion of the meta information), and optionally one or more images captured by imaging components within the head portion 205. The examination results 185 identify whether the patient has Acute Otitis Media (AOM) or Otitis Media with effusion (OME) for example. For another embodiment, the examination results 185 may include diagnostic metrics 180 (and/or corresponding images) for comparison with heuristics associated with prior finding of AOM (or OME) and/or diagnostic metrics (and/or corresponding images) of healthy middle ears.

For this embodiment, the base station 160 includes a display screen 260 to display the examination results 185 described above. For this embodiment, the display screen 260 may be part of an electronic device, such as a tablet running a particular operating system (e.g., Microsoft® Windows®, Google® Android® or Apple® IOS®, etc.), a desktop computer, a cellular smartphone, or the like. The base station 160 may include one or more components for communications with spectral illumination light sources (e.g., LEDs), with amplification logic for integrating with photodiodes being part of the OS data acquisition components 155, and/or cameras.

According to another embodiment of the disclosure, the OS components, including the OS data acquisition components 155 and the OS analytic components 156 for example, may be collectively deployed in both the diagnostic tool 150 and the base station 160. In particular, the diagnostic tool 150 includes the OS data acquisition components 155, which is configured to measure the reflectance spectra by converting the measurement spectra into an acceptable format (OS data 154). However, the OS analytic components 156 may be implemented within the diagnostic tool 150 or within the base station 160 (represented as an optional feature by dashed lines), where the OS analytic components 156 are configured to analyze the OS data 154 to determine characteristics (e.g., a parameter or combination of parameters such as shape, magnitude, etc.) of the reflectance spectra and compute the diagnostic metrics 180.

As a result, when the OS analytic components 156 are implemented within the base station 160, the OS data 154 may be provided to the base station 160 through the wired interface 225 and cable 235. The cable 235 would be shielded as described above. Reliance on the base station 160 for generation of the examination results 185 may allow for the installation of OS analytic components 156 with greater processing and/or storage capability (due to greater installation area, greater thermal management, etc.).

C. Wireless Point-of-Care (POC) System

Referring now to FIG. 3A, an illustrative embodiment of the POC system 110 with wireless connectivity between the base station 160 and the diagnostic tool 150 is shown. As illustrated in FIG. 2A and described above, the diagnostic tool 150 may be implemented as a non-invasive otoscope for diagnosing health conditions, such as ear conditions including different types of OM. The diagnostic tool 150 includes the head portion 205, the removable (and replaceable) speculum 210, and the handle 215, which includes the measure button 220. In particular, the diagnostic tool 150 has been modified to include optical spectroscopy (OS) components 155/156 as described above; however, the diagnostic tool 150 includes wireless interface 300, namely logic that supports the wireless transmission of the OS data 154 or examination results 185 (and/or image data), depending on the deployment, to the base station 160 over a wireless interconnect 310. The transmission may be automatic (push) or in response to a request “REQ” message (pull). The wireless interconnect 310 may be configured in accordance with radio frequency (RF) signalling standards, Bluetooth signalling standards, cellular signalling standards, or the like.

Referring now to FIG. 3B, an illustrative embodiment of the POC system 110 with wireless connectivity between the diagnostic tool 150 and a cellular telephone (smartphone) 350 is shown. As illustrated in FIGS. 2A and 3A and described above, the diagnostic tool 150 may be implemented as a non-invasive otoscope for diagnosing health conditions, such as ear conditions including different types of OM. The diagnostic tool 150 includes the wireless interface 300 that supports the wireless transmission of either the OS data 154 or the examination results 185 (depending on the deployment) and/or images captured by imaging components within the diagnostic tool 150 to the cellular telephone 350 over the wireless interconnect 310.

For one embodiment of the disclosure, the cellular telephone 350 may be configured with the OS analytic components 156 or software with functionality provided by the OS analytic components 156. For instance, as an illustrative example, the OS analytic components 156 may include one or more software applications stored within memory and, when executed, are configured to analyze the OS data 154 and generate the diagnostic metrics 180 therefrom. Herein, the OS analytic components 156 may collect the diagnostic metrics 180 to produce the examination results 185 and render one or more objects on a screen 355 of the cellular telephone 350 to identify whether the examination results 185 determined the tissue under examination is healthy or infected (e.g., presence of AOM, presence of group A streptococcus “GAS,” etc.). Additionally, or in the alternative, the examination results 185 may be transmitted to a network (e.g., cloud network 370, also represented as cloud network 130₂ of FIG. 1) for further analysis and/or storage.

As another embodiment of the disclosure, the cellular telephone 350 may be configured with logic, namely one or more software applications stored within memory and, when executed, are configured to transmit the received OS data 154 to OS analytic components 156 deployed as part of a cloud service 380 offered by a public or private cloud network 370 (e.g., virtual compute engines, virtual data stores, etc.). Herein, the analysis of the OS data 154 occurs within the cloud network 370, where the resultant examination results 185 may be stored therein and accessible to the cellular telephone 350 and/or a clinician with access to the examination results 185 associated with a particular patient.

As yet another embodiment of the disclosure, the cellular telephone 350 may be configured with logic, namely one or more software applications stored within memory and, when executed, are configured generate and render one or more objects on the screen 355 to identify whether the examination results 185, determined at the diagnostic tool 150, indicate that the tissue under examination is healthy or infected. Also, the software application(s) may control storage of the examination results 185.

III. General Embodiments of the Diagnostic Tool

Referring to FIG. 4A, a first illustrative embodiment of a fragmentary cross-sectional perspective of the diagnostic tool 150 for diagnosing health conditions, such as ear conditions including different types of Otitis Media (OM), is shown. The diagnostic tool 150 includes the head portion 205 that features a light conveyance component 410 including a fixed, disc-shaped connection subcomponent 415 and a conical-shaped subcomponent 418. The removable (and replaceable) speculum 210 is sized for placement over a substantial portion of the conical-shaped subcomponent 418. Connected to the head portion 205, the handle 215 includes the measure button 220 and a wired (or wireless) interface 225 or 300. In particular, the diagnostic tool 150 may operate as the otoscope, but it has been modified to include at least OS data acquisition components 155 for emitting light into a portion of a body cavity (e.g., ear canal) via the light conveyance component 410 and the removable speculum 210, receiving light returning from the body cavity, measuring reflectance intensity from a spectra of the returned light (reflectance spectra), uploading the analysed data associated with the reflectance spectra (e.g., OS data 154 and/or diagnostic metrics 180, etc.) for storage as part of a patient's digital medical record. At certain times, the diagnostic tool 150 may receive software updates and/or heuristic data to improve operability of the OS analytic components 156 and/or OS data acquisition components 155 to better assess a presence of a certain ailment through OS-based examinations.

Herein, the diagnostic tool 150 may include a visualization optics 400, such as an optical lens 405 to enable a user to visually inspect tissue within a portion of a body cavity (e.g., ear cavity). The visual review by the clinician along with the automated, OS-based examination may reduce the frequency of false positives and/or false negatives. The analysis of the OS data 154 may be conducted by the OS analytic components 156 within the diagnostic tool 150.

In the alternative, as shown in FIG. 4B, the OS analytic components 156 may be deployed within a removable smart card 420, which is communicatively coupled to a card adapter 425 upon insertion via slot 422. The card adapter 425 may be communicatively coupled to other components within the handle 215 of the diagnostic tool 150. According to one embodiment, the combination of the OS data acquisition components 155 and OS analytic components 156, deployed within the removable smart card 420, generates OS data 154 or examination results 185, which identify the health of a portion of a tissue under inspection (e.g., portion of the middle ear). These results may include, but are not limited or restricted to (i) a presence or absence of OM and/or (ii) presence or absence of middle ear effusion, (iii) presence or absence of otitis externa, (iv) probability of developing an ear condition, and/or (iv) a computed hemoglobin level.

Referring back to FIG. 4A, the OS data acquisition components 155 may include an illumination assembly 430, a detection assembly 432, and an optional temperature sensing assembly (not shown) to measure temperature on or around a surface of the portion of the ear canal. As shown, the illumination assembly 430 may include one or more spectral light sources (hereinafter, “spectral light source 430”) positioned to emit light through corresponding conduits 450 (one of the conduits 230 of FIG. 2A) to illuminate the ear canal with incident light at predetermined wavelengths. The predetermined wavelengths are selected to identify specific conditions such as water (900 nm -1700 nm wavelength), hemoglobin (400 nm -1000 nm wavelength). Additionally, the detection assembly 432 may include one or more light detection elements (hereinafter, “light detection element 432”), which are configured to receive light returning from the ear canal via corresponding conduits 452 in the speculum 210 and measure a reflectance intensity as a function of the predetermined wavelengths to diagnose a presence of OM. For this embodiment, the light detection element 432 may include one or more photodiodes with distinct spectral sensitivities, where the spectral light source 430 may feature one or more LEDs having spectral distributions that overlap each photodiode sensitivity and may be illuminated concurrently (i.e. at least partially overlapping in time). Concurrent illumination may achieve reduced measurement time without crosstalk.

The measure button 220 is situated to protrude from an outer surface of the handle 215, where the measure button 220 may be positioned at any location on the diagnostic tool 150. Upon depression, the measure button 220 triggers activation of the spectral light source 430 and/or the light detection element 432. For instance, as an embodiment of the disclosure, the spectral light source 430 may feature one or more of light-emitting diodes (LEDs), multi-LED chip, laser diodes, or vertical-cavity surface-emitting laser (VCSELs). The light detection element 432 may feature one or more photodiodes, photomultiplier tubes (PMT), complementary metal-oxide-semiconductor (CMOS) arrays, charge-coupled device (CCD) arrays, spectrometers, and/or fabry-perot interferometers. Each light detection element 432 is configured with a central wavelength that matches a specific wavelength of spectral light produced by a corresponding spectral light source 430 (e.g., LED of the multi-chip LED). In some embodiments, the central wavelength may be predetermined and selected from a range of wavelengths between about 400 nm and 2000 nm.

According to one embodiment of the disclosure, at least two photodetectors with complementary spectral sensitivities are utilized to span the required spectral range. In some of these embodiments, the complementary sensitivities may partially overlap. For example, a first photodiode, being part of the light detection element 432, may be deployed as a silicon photodiode that is sensitive to a spectral (wavelength) ranging from approximately 400 nm-1100 nm. A second photodiode, being part of the light detection element 432, may be deployed as an InGaAs photodiode that is sensitive to a spectral range of approximately 900 nm-1700 nm.

According to another embodiment of the disclosure, a single photodiode may be implemented as the light detection element 432 in lieu of a plurality of photodiodes. For this embodiment, this silicon photodiode may be configured with a spectral sensitivity between 400-1100 nm. As an optional feature, the single photodiode may be “enhanced” to either (1) extend the sensitive range or (2) increase the output signal of the photodiode within the sensitive range. More specifically, the photodiode may be enhanced in the infrared wavelength, which enables light sensitivity to detect light exceeding 1100 nm wavelength. Additionally, or in the alternative, the photodiode may be enhanced in the visible/UV wavelengths, which improve sensitivity of detected light at wavelengths less than 400 nm.

Different matter within the ear canal may constitute a set of optical contrasts, each contributing to the magnitude of the reflectance spectra. For example, hemoglobin species contribute to erythema (redness), lipid species constitute wax and create pronounced spectral differences in blue/green color wavelength indices, water content of middle ear effusions create contrast in the infrared index, etc. Water is generally present in most ear tissue and may create contrasts related to inflammation, allergies, or other health condition. Hence, some or all of these ear characteristics may contribute to changes in reflectance spectra due to their optical properties (e.g. scattering and absorption). These optical properties may vary in a consistent way with different types of ear pathology. From these optical properties, the OS analytic components 156 is responsible for analyzing the OS data and determine whether the patient has a particular ailment (e.g., AOM, strep throat, etc.).

Referring still to FIG. 4A, the OS analytic components 156 includes a controller 460 and a memory 470. In some embodiments, being supplied power from an internal power source (not shown), the controller 460 is configured to analyze the OS data by at least comparing (i) characteristics (e.g., the diagnostic metrics data) determined from data produced from the reflectance spectra pertaining to the reflected light collected by the light detection element 432 to (ii) data associated with characteristics determined from data associated with known reflectance spectra associated with healthy and/or unhealthy ear conditions (referred to as “reference metric distributions”). Each of the reference metric distributions may be determined based on machine learning or heuristics that considers data from one or more prior analysis of ear conditions for that particular patient or patients previously evaluated and having the same metrics. The reference metric may be parametric or non-parametric in nature. The reference metric distributions may be stored locally in the memory 470 or downloadable from a remote database.

While the diagnostic tool 150 is directed to detection of types of otitis media (e.g., AOM), when implemented with visualization optics 400, the diagnostic tool 150 may include one or more optic-based illumination light sources 480 (e.g., brightfield light sources) and one or more spectral light sources 430 (e.g., multiple LEDS). Herein, the brightfield light sources 480 are oriented to illuminate the ear for visual evaluation, where a plurality of optical diffusers (not shown), may be positioned in-line with the brightfield light sources 480 (e.g., within channel 454) to diffuse light prior to entry into the ear canal.

According to another embodiment as shown in FIG. 4B, the diagnostic tool 150 features the removable smart card 420. According to one embodiment of the disclosure, the smart card 420 is adapted for communicative coupling with the card adapter 425, which enables communications with the controller 460. For this embodiment, the OS analytic components 156 include scan monitoring logic 490 to monitor a scan count, which represents a number of prepaid measurement scans to assess health conditions of the patient though analysis of reflectance spectra within the ear canal.

Responsive to depression of the measure button 220 after the speculum 210 is inserted into a patient's ear canal, the scan count is recovered by the scan monitoring logic 490 from data store 495 maintained by the smart card 420 to determine that there are sufficient pre-paid scans available before the scan conducted. If so, the scan count maintained by the smart card 420 is decremented by scan monitoring logic 419 and the spectral light source 430 is activated by the controller 460 to emit incident light into the middle ear cavity, while the light detection element 432 is activated to collect returning light after reflection and/or refraction of the emitted light. The returning light may be measured by the OS components 155/156, along with the controller 460, to determine health-related metrics for the patient. Although not shown, it is contemplated that a notification element may be positioned on the head portion 205 or handle 215, which is visible to a user and identifies either the number of pre-paid scans available or the number of pre-paid scans has decreased to be equal to or less than a prescribed count value (e.g., 0-to-5 pre-paid scans).

According to another embodiment of the disclosure, the smart card 420 may be adapted for communicative coupling with the card adapter 425; however, in lieu of the controller 460 conducting the analysis of the OS data 154 associated with the reflectance spectra as described above, the smart card 420 may include logic that not only maintains the scan count, but also performs such analysis in lieu of (or in combination with) the controller 460. As a result, software upgrades for the generation, recovery and/or analysis of the diagnostic data may be loaded onto the local data store 495 on the smart card 420.

Referring to FIG. 5A, a first illustrative embodiment of a camera-based diagnostic tool 150 is shown. Herein, the camera-based diagnostic tool 150 features OS data acquisition components 155 arranged within the head portion 205. The OS data acquisition components 155 include a multi-LED chip 510, which controls emission of light from multiple spectral light sources (e.g., LEDs) 515 through the conduits 230 (e.g., channels, tubes, etc.) integrally formed within the light conveyance component 410. The emitted light for each LED 515 may possess a different predefined wavelength (e.g., a first LED associated (e.g. peak, or centroid) with a wavelength of approximately 500 nm; a second LED associated with a wavelength of approximately 577 nm; a third LED associated with a wavelength of approximately 970 nm, etc.). Furthermore, the LEDs 515 may operate in a serial or concurrent manner.

The OS data acquisition components 155 further include one or more photodetectors 525, each is aligned with a corresponding conduit 452 formed within the interior of the light conveyance component 410 to receive reflected light passing through the opening 232 of the speculum 210 and an opening 530 with the conical-shaped subcomponent 418. It is contemplated that a gradient index lens 535 may be positioned in-front of and aligned with each or some of the photodetectors 525. As shown, one of the gradient index lens 535 may be positioned with the conduit 452 utilized by a corresponding photodetector 525. The conduits 452 may feature different diameter segments to secure the gradient index lens 535, and thus, this type of conduit 452 may be different than the conduit 450 utilized by the spectral light sources 515. Each of the photodetectors 525 is configured to detect a different reflectance spectra and generates OS data 154 to be provided to a controller (not shown) deployed within the diagnostic tool 150.

Additionally, the camera-based diagnostic tool 150 includes visualization logic 400 in combination with imaging logic 560. The virtualization logic 400 allows a clinician 545 looking through the magnifying lens 550 and a viewing aperture 552 aligned with the opening 232 of the speculum 210 and the opening 530 within the conical-shaped component 418 of the light conveyance component 410. The imaging logic 560 includes a beam splitter 570 and one or more cameras 580₁-580_{L} (L>1). In particular, the returned (reflected) light is received by the beam splitter 570, which passes a portion of the returned light from the body cavity to the clinician 545 for viewing via magnifying lens 550 and a remainder of the returned light to the cameras 580₁-580_{L}. The ratio of the returned light provided to the clinician 545 and the cameras 580₁-580_{L} may be static (e.g., 50-50%; 80-20%, etc.) or dynamically set.

Herein, each of the cameras 580₁-580_{L} may capture image(s) of the tissue within the body cavity under analysis (e.g., tissue in middle ear), where the image of the ear, may also be visible to the clinician 545. The cameras 580₁-580_{L} may support different image types. For example, a first camera 580₁ (first photo sensor) includes a spectral filter on the red, green and blue pixels in order to pass passing red, green and blue light elements. Similarly, a second camera 580₂ may not include any color-based filters, where the cameras may be more sensitive to light of a specific wavelength (e.g., 970 nm to contrast the presence of water (middle ear infusion), or 577 nm to contrast water from hemoglobin). And so, the cameras 580₁-580_{L} would capture a set of images (e.g., color and/or black/white), to enhance the contrast of those images. Also, these captured images, notably black/white images, may be used to calculate shape or, for example, bulging of the tympanic membrane. In another embodiment, one or more cameras may be combined with structured illumination to assess the shape of the tympanic membrane.

Referring to FIGS. 5B-5C, a second illustrative embodiment of a camera-based diagnostic tool 150 is shown. Herein, the camera-based diagnostic tool 150 features OS data acquisition components 155 arranged within the head portion 205 as described above. However, in lieu of usage of the beam splitter 570 as shown in FIG. 5A, the imaging logic 560 may include one or more cameras 580₁-580_{L} positioned around a perimeter 585 of the connection subcomponent 415, as shown in FIG. 5C. Stated differently, the configuration of the OS data acquisition components 155 (e.g., multi-LED chip 510, photodetectors 525, etc.) would be similar as shown in FIG. 5A, but the one or more cameras 580₁-580_{L} would be oriented in a manner similar to the photodetectors 525. Each of the cameras 580₁-580_{L} would be oriented with a line-of-sight to the tissue under examination via conduits 456, where the photodetectors 525 may or may not be in a line-of-sight orientation. The cameras 580₁-580_{L} may include one or more silicon cameras, one or more InGaAs cameras, or any combination thereof.

Referring to FIG. 5D, a third illustrative embodiment of a camera-based diagnostic tool 150 is shown. Herein, the camera-based diagnostic tool 150 features OS data acquisition components 155 arranged within the head portion 205 of the diagnostic tool 150. As in FIGS. 5A-5C, the OS data acquisition components 155 may include a multi-LED chip 510, which controls emission of light from multiple spectral light sources (e.g., LEDs) 515 through one or more conduits 450 formed within an interior of the light conveyance component 410. As described above, the emitted light for each LED 515 may possess a different predefined wavelength and operate in a serial or concurrent manner.

The OS data acquisition components 155 further include one or more photodetectors 525, which are aligned with one or more corresponding conduits 452 formed within the interior of the light conveyance component 410 to receive light rays from a reflected light received at the opening 232 of the speculum 210 and the opening 530 of the light conveyance component 410. Similar to FIG. 5A, the gradient index lens 535 may be positioned in-front of and aligned with each of the photodetectors 525, and perhaps inserted within the corresponding conduit 452. However, in lieu of virtualization logic 400 as shown in FIG. 5A, the camera-based diagnostic tool 150 includes one or more cameras 580₁-580_{L} (e.g., camera 580₁) centralized within a prescribed conduit 590 to capture image(s) of the tissue within the body cavity under examination (e.g., tissue in middle ear) for display on a display screen 595 positioned on the back end of the head portion 205. Additionally, or in the alternative, the image(s) may be stored with local memory of the diagnostic tool 150 to allow for subsequent transmission via the wired or wireless interface to the base station (not shown).

Referring to FIG. 5E, a fourth illustrative embodiment of the diagnostic tool 150 with visualization logic 400 is shown. Herein, the diagnostic tool 150 features the OS data acquisition components 155 arranged within the head portion 205 of the diagnostic tool 150 as described above. However, in lieu of cameras 580₁-580_{L} being deployed within the diagnostic tool 150, the diagnostic tool 150 includes the visualization logic 400 including the magnification lens 550 with the conduit 590 aligned with openings 232 and 530 to provide a line-of-sight to the tissue within the body cavity under inspection. The multi-chip LED 510 and photodetectors 525 are deployed in a manner similar to any of FIGS. 5A-5D.

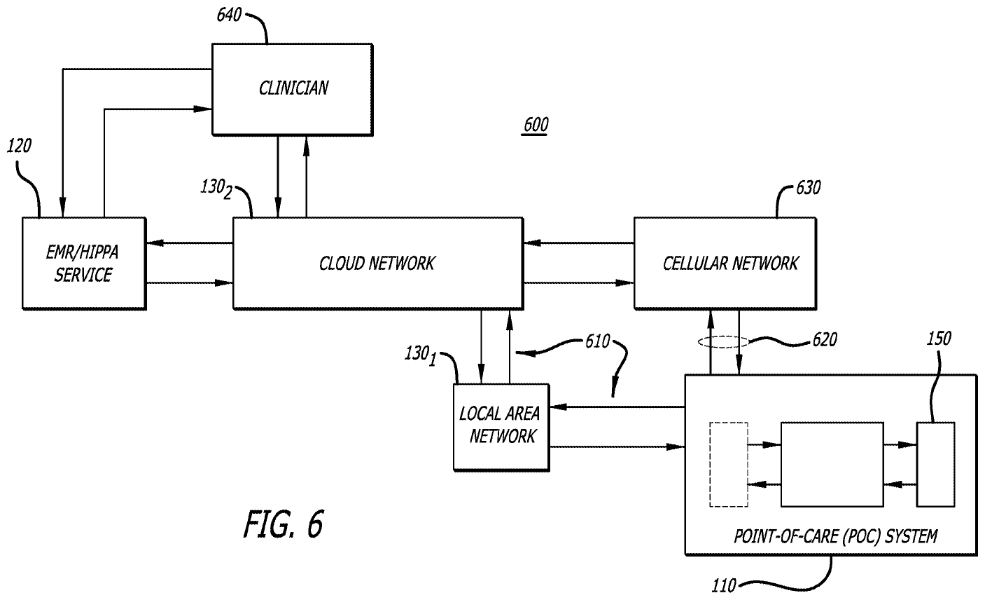

Referring now to FIG. 6, a second exemplary embodiment of the health management system 600 is shown. The health management system 600 comprises a point-of-care (POC) system 110 that is configured to analyze the condition of tissue under diagnosis (e.g., presence of AOM within a middle ear of a patient) without components that provide for visualization optics. As in FIG. 6, the POC system 110 is communicatively coupled to the local area network 130₁ or the cloud network 130₂, which may be accessible through radio-frequency (RF) transmission or cellular transmissions 620 via cellular network 630.

As shown, electronic medical records service 120 may be communicatively coupled to the cloud network 130₂, where the service 120 maintains patient electronic file histories in compliance with the Health Insurance Portability and Accountability Act (HIPAA). As a result, the transmission of the OS data 154 or examination results 185 from the POC system 110 to the cloud network 130₂ would need to be accompanied by a patient identifier (e.g., social security number, clinician patient number, etc.). The patient identifier may be used by a clinician 640 to locate the examination results computed by either the POC system 110 or compute engines within the cloud network 130_{2.}

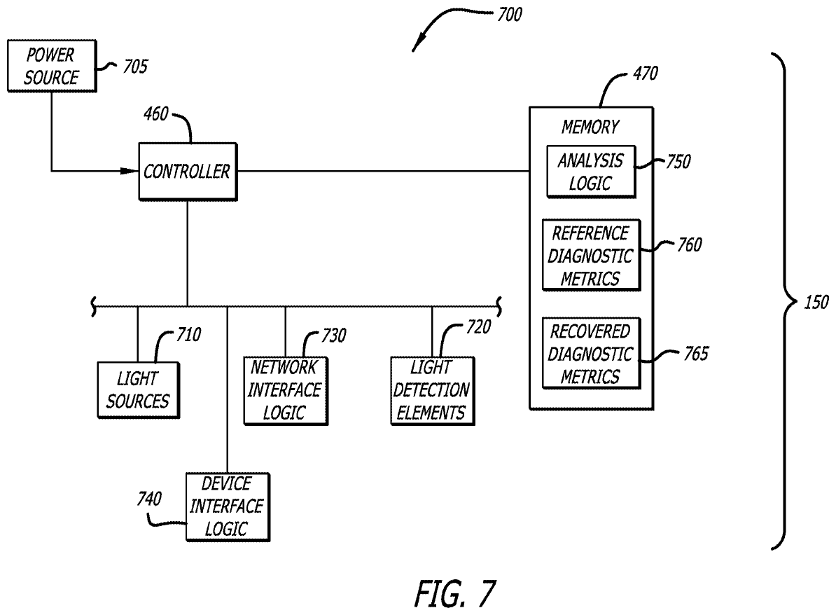

Referring to FIG. 7, an exemplary embodiment of a logical architecture 700 of the diagnostic tool 150 of FIG. 1 is shown. Herein, the logical architecture 700 includes a power source 705 that provides power to one or more components deployed within the diagnostic tool 150. Example of components deployed within the diagnostic tool 150 may include, but are not limited or restricted to the controller 460, memory 470, light sources 710 (e.g., spectral light sources 432, etc.), light detection elements 720 (e.g., photodetectors 525, etc.), network interface logic 730, and/or device interface logic 740.

Herein, the controller 460 is configured to conduct analytics on OS data associated with the returned, reflected light (reflectance spectra), which is detected by the light detection elements 720 from light emitted from the light sources 710. For this embodiment of the disclosure, the controller 460 corresponds to data processing circuitry, which may include, but is not limited or restricted to any type of hardware processor (e.g., microprocessor with one or more processor cores, a digital signal processor, a programmable gate array, a microcontroller, an application specific integrated circuit “ASIC”, etc.).

As shown in FIG. 7, the controller 460 is communicatively coupled to the memory 470, which maintains analysis logic 750, namely logic that controls operability of the diagnostic tool 150 including conducting analytics between diagnostic metrics associated with known reflectance spectra associated with healthy and/or unhealthy ear conditions (e.g., reference diagnostic metrics 760) and diagnostic metrics 765 associated with recovered reflectance spectra from the emitted light during the current analysis. For instance, as an illustrative example, the analytics may involve at least a comparison between (i) diagnostic metrics 765 determined from data produced from the reflectance spectra pertaining to the reflected light collected by the light detection elements 720 based on emitted light from the light sources 710 and (ii) one or more reference diagnostic metrics 760. Herein, each reference diagnostic metric 760 may be determined through machine learning or heuristics that considers data from one or more prior analysis of ear conditions for that particular patient or patients previously evaluated and having the same metrics. The reference diagnostic metrics 760 may be stored locally in the memory 470 or accessible from remote storage. In some embodiments, the local memory 470 may be physically replaceable by the user. In some embodiments, the local memory 470 may be embedded in the removable speculum 210. In other embodiments, the local memory 470 may take the form of a removable smart card.

The network interface logic 730 is configured to operate as a transceiver to receive inputs (e.g., updates to the analysis logic 750, additional or substitute reference diagnostic metrics 760, etc.) and to output the examination results 185 to a base station (or cellular telephone) for subsequent display and/or storage. The device interface logic 740 is configured to provide connectivity between the controller 460 and various types of input/output (I/O) devices (e.g., measure button 220, etc.) and ancillary devices (e.g., removable smart card 420, etc.).

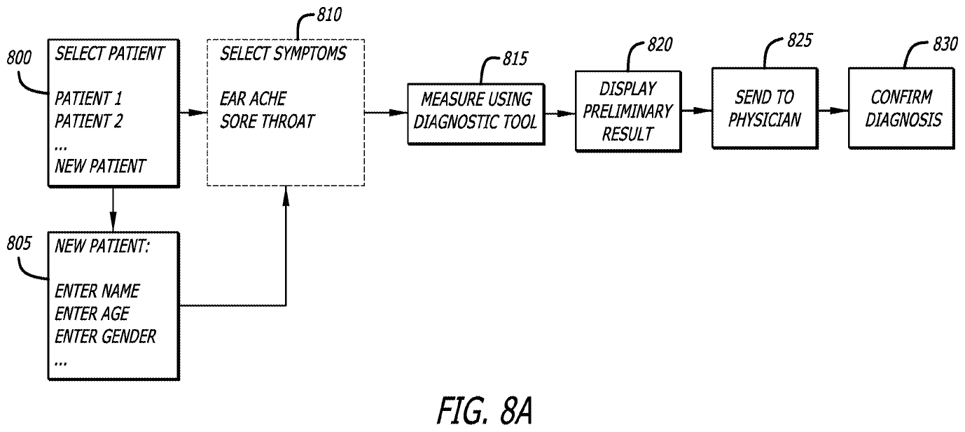

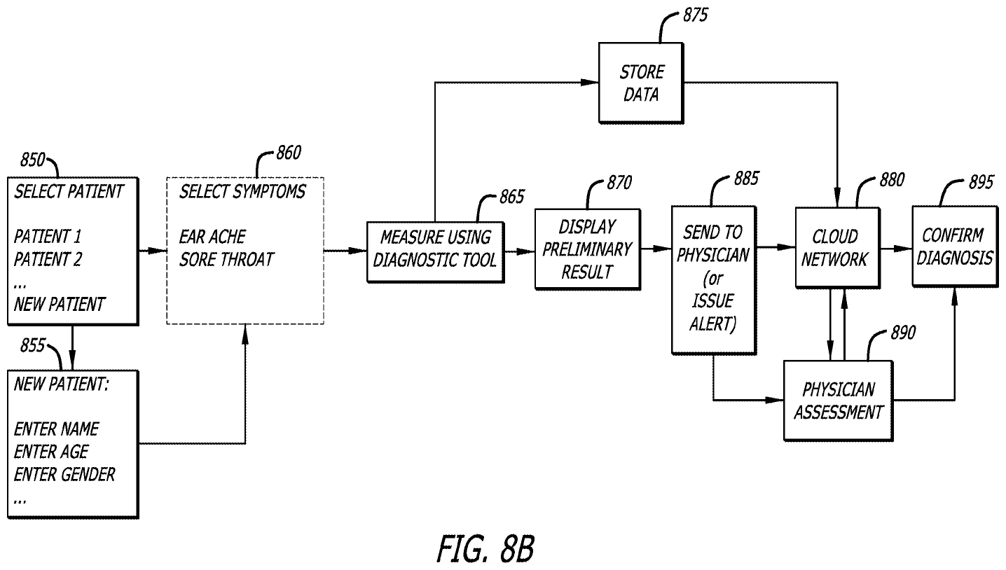

Referring to FIG. 8A, a first exemplary embodiment of the operations of the diagnostic tool 150 operating within the health management system 600 of FIG. 6 is shown. Herein, when registered, the patient associated with an upcoming diagnostic event may be selected (block 800). For example, the patient may be registered with the diagnostic tool, where the name/age/gender of the patient are entered and the patient may be selected by setting one or more switches, moving a cursor to select the patient's name displayed on a LCD screen provided on the diagnostic tool, or the like. It is contemplated that other information, besides or in addition to name/age/gender, may be used to identify the patient (e.g., social security number, etc.). Otherwise, the patient will need to be registered with the diagnostic tool (block 805).

After the patient is selected, as an optional feature, the user may select the symptoms for which the diagnosis is to be directed (block 810). For example, the symptoms may include an ear ache, which causes the diagnostic tool to load analysis logic that is directed to detect the presence of Otitis Media (OM) such as Acute Otitis Media (AOM). Where the symptoms are sore throat, the diagnostic tool to load analysis logic that is directed to detect the presence of group A streptococcus (GAS), which may be used to denote the presence of strep throat. However, this feature would not be deployed in the case where the diagnostic tool is directed to a single type of ailment (e.g., detection of Otitis Media conditions such as AOM).

Thereafter, responsive to depression of the measure button, the diagnostic tool performs a measurement by emitting light into a body cavity including the potentially unhealthy tissue and collecting reflected light returned from the body cavity (reflectance spectra). The reflectance spectra is used to determine the OS data, which may be analysed by the diagnostic tool (or a base station) located proximate to the diagnostic tool to compute diagnostic metrics that collectively form the examination result (block 815). The examination results may be displayed at the diagnostic tool or displayed at a separate device such as at a display communicatively coupled to the base station (block 820).

The examination results may be stored with a data store maintained within the diagnostic tool and/or transmitted to the clinician directly or indirectly through placement of the examination results within an electronic medical record associated with the patient that is accessible by the clinician (block 825). Thereafter, the clinician is provided secured access to the examination results, when the clinician has access to the patient's electronic medical record, and as a result, the clinician may confirm the diagnosis computed by the diagnostic tool and/or issue a prescription for medicine (e.g., antibiotics, etc.) to address the ailment associated with the unhealthy tissue (block 830).

Referring to FIG. 8B, a second exemplary embodiment of the operations of the diagnostic tool 150 operating within the health management system 600 of FIG. 6 is shown. Herein, different than FIG. 8A, the diagnostic tool also stores the diagnostic metrics (including any images) associated with the examination results into a private virtual network established within the cloud network. Access to the stored data is controlled in accordance with HIPAA standards. The clinician may access the examination results over a secure communication path (e.g., encrypted, secure socket layer (SSL), etc.) to retrieve the examination results to confirm the findings made by the diagnostic tool.

More specifically, when registered, the patient associated with an upcoming diagnostic event may be selected (block 850). For example, the patient may be registered with the diagnostic tool, where selection of the patient may be accomplished by setting one or more switches, moving a cursor to select the patient's name displayed on a LCD screen provided on the diagnostic tool, or the like. Otherwise, the patient will need to be registered with the diagnostic tool (block 855). After the patient is selected, as an optional feature, the user may select the symptoms for which the diagnosis is to be directed toward (block 860). This selection may cause the loading of different software or activation of different OS components. However, where the diagnostic tool is directed to a single type of ailment (e.g., detection of Otitis Media conditions such as AOM), the selection of symptoms is not necessary.

Thereafter, responsive to depression of the measure button, the diagnostic tool performs a measurement by emitting light into a body cavity including the potentially unhealthy tissue and collecting reflected light returned from the body cavity (reflectance spectra). The reflectance spectra is used to determine the OS data, which may be analysed by the diagnostic tool (or a base station) located proximate to the diagnostic tool to compute diagnostic metrics that collectively form the examination result (block 865). The examination results may be displayed at the diagnostic tool or displayed at a separate device such as at a display communicatively coupled to the base station (block 870). Additionally, some or all of the diagnostic metrics forming the examination results are uploaded into a secure storage location within the cloud network (blocks 875-880). For example, the diagnostic metrics may be uploaded into a private virtual network established within the cloud network, where the diagnostic metrics may be accessed by a clinician only through a secure access mechanism that is compliant with federal and state regulations as to patient medical records. Access to the stored data is controlled in accordance with HIPAA.

Herein, the clinician may receive the diagnostic metrics directly, or may receive an alert indicating that diagnostic metrics are awaiting review by the clinician (block 885). The alert may include a link to the diagnostic metrics, where authentication is necessary for access to the content associated with the diagnostic metrics is provided. Also, the alert may identify the patient and time that the diagnostic metrics were computed to assist the clinician in confirming the findings made by the diagnostic tool occurred within a prescribed period of time to ensure that the diagnostic metrics are timely. Thereafter, the clinician is provided secured access to the examination results, where the clinician may confirm the diagnosis computed by the diagnostic tool and/or issue a prescription for medicine (e.g., antibiotics, etc.) to address the ailment associated with the unhealthy tissue (blocks 890-895).

In the foregoing description, the invention is described with reference to specific exemplary embodiments thereof. However, it will be evident that various modifications and changes may be made thereto without departing from the broader spirit and scope of the invention as set forth in the appended claims.

What is claimed is:

1. A point-of-care system, comprising:
a diagnostic tool for use in diagnosing health conditions for a tissue under analysis, the diagnostic tool includes a handle and a head portion with a speculum and optical spectroscopy (OS) data acquisition components, the OS data acquisition components are configured to (i) emit light toward the tissue under analysis, (ii) receive light reflected at least in part from the tissue under analysis based on the emitted light, and (iii) determine reflectance spectra associated with the received light; and
a base station communicatively coupled to the diagnostic tool, the base station includes a display screen,
wherein the diagnostic tool or the base station includes analytic components that are configured to (i) generate diagnostic metrics that include characteristics of the reflectance spectra and (ii) compare the characteristics of the reflectance spectra to data associated with characteristics of known reflectance spectra associated with at least one of healthy tissue or tissue of patients exhibiting health conditions.

2. The point-of-care system of claim 1, wherein the analytic components are deployed within both the diagnostic tool and the base station.

3. The point-of-care system of claim 1, wherein the OS data acquisition components of the diagnostic tool include
an illumination assembly including at least one or more spectral illumination light sources, each of the one or more spectral illumination light sources is arranged within the head portion to emit light of a particular central wavelength through an opening of the speculum toward the tissue under analysis; and
a detection assembly including one or more light detection elements, each of the one or more light detection elements is arranged to collect the received light propagating through the opening of the speculum.

4. The point-of-care system of claim 3, wherein each of the one or more light detection elements corresponds to one or more of the spectral illumination light sources to detect light with the particular central wavelength ranging between 400 nanometers to 2000 nanometers.

5. The point-of-care system of claim 1, wherein the characteristics of the known reflectance spectra may be determined based on machine learning or heuristics based on prior analyses of the tissue under analysis that includes tissue within an ear canal including a tympanic membrane or middle ear.

6. The point-of-care system of claim 1, wherein the diagnostic tool further comprises a set of imaging components installed within the head portion of the diagnostic tool to capture one or more images associated with a body cavity including the tissue of analysis.

7. The point-of-care system of claim 6, wherein wavelengths of the emitted light are selected so that greater imaging contrasts within an image generated by the one or more imaging components occur for different tissue conditions.

8. The point of care system of 6 further comprises visualization optics that, when operating in combination with the set of imaging components, enables simultaneous image capture and visualization.

9. The point-of-care system of claim 6, wherein the set of imaging components are configured to capture a degree of distention of an eardrum to detect a bulging or retracting tympanic membrane.

10. The point-of-care system of claim 6, wherein the diagnostic tool and/or the base station further comprises interface components configured for input of qualitative metrics to provide context to the image generated by the set of imaging components, the qualitative metrics include data related to a patient associated with the tissue under analysis.

11. The point-of-care system of claim 1, wherein the analytic components, deployed within the diagnostic tool, include a controller and a memory that includes reference diagnostic metrics including the data associated with the characteristics of the known reflectance spectra, the controller is configured to conduct a comparison between the diagnostic metrics and the reference diagnostic metrics.

12. The point-of-care system of claim 1, wherein the analytic components, deployed within the base station, include a controller and a memory that includes reference diagnostic metrics including the data associated with the characteristics of the known reflectance spectra, the controller is configured to conduct a comparison between the diagnostic metrics and the reference diagnostic metrics.

13. The point-of-care system of claim 1, wherein the base station includes image rendering logic to convert an optical image captured by the set of imaging components operating as a camera into an image or the series of images for display on the display screen of the base station.

14. The point-of-care system of claim 6, wherein the base station is communicatively coupled to the diagnostic tool through a cable including a plurality of interconnects, the plurality of interconnects includes a first subset of interconnects conveying information associated with the diagnostic metrics shielded from a second subset of interconnects conveying data associated with the one or more images captured by the set of imaging components.

15. A diagnostic tool for use in diagnosing health conditions for a tissue under analysis, the diagnostic tool comprising:
a handle; and
a head portion including a light conveyance component including a first subcomponent including a plurality of conduits and a second subcomponent oriented in a conical shape, the first subcomponent being configured to include (1) one or more imaging components aligned with a first set of one or more conduits of the plurality of conduits, (2) one or more spectral illumination light sources aligned with a second set of one or more conduits of the plurality of conduits, each of the one or more spectral illumination light sources is configured to emit light of a particular central wavelength through a corresponding conduit of the second set of one or more conducts for propagation through the second subcomponent toward the tissue under analysis, and (3) one or more light detection elements aligned with a third set of one or more conduits of the plurality of conduits, each of the one or more light detection elements is configured to collect the received light propagating through an opening of the second subcomponent and a conduit of the third set of one or more conduits and determine reflectance spectra associated with the received light.

16. The diagnostic tool of claim 15 further comprising:
analytic components that are configured to (i) generate diagnostic metrics that include characteristics of the reflectance spectra, (ii) compare the characteristics of the reflectance spectra to data associated with characteristics of known reflectance spectra associated with at least one of healthy tissue or tissue of patients exhibiting health conditions to generate results for upload to a cloud network via a network interface; or
an interface to provide communicative coupling to a base station remotely located from the diagnostic tool, the base station includes analytic components that are configured to (i) generate the diagnostic metrics that include the characteristics of the reflectance spectra, (ii) compare the characteristics of the reflectance spectra to the data associated with characteristics of the known reflectance spectra associated with at least one of healthy tissue or tissue of patients exhibiting health conditions to generate the results for upload to the cloud network via the network interfaces.

17. The diagnostic tool of claim 16 being communicatively coupled to the base station including a display to render projections of a comparison of the characteristics of the reflectance spectra and the data associated with characteristics of known reflectance spectra to determine whether the tissue under analysis features an ailment.

18. The diagnostic tool of claim 15, wherein the one or more imaging components are configured to capture a degree of distention of an eardrum to detect a bulging or retracting tympanic membrane.

19. The diagnostic tool of claim 16, wherein the analytic components, deployed within the diagnostic tool, include a controller and a memory that includes reference diagnostic metrics including the data associated with the characteristics of the known reflectance spectra, the controller is configured to conduct a comparison between the diagnostic metrics and the reference diagnostic metrics.

20. The diagnostic tool of claim 15 further comprising:
visualization optics installed to enable visual inspection of the tissue under analysis to be conducted concurrently with the emitting of the light by any of the one or more spectral illumination light sources or the collecting of the received light based on the emitted light by any of the one or more light detection elements.

21. A diagnostic tool for use in diagnosing health conditions for a tissue under analysis, the diagnostic tool comprising:
a handle;
a head portion including a light conveyance component including a first subcomponent including a plurality of conduits and a second subcomponent oriented in a conical shape, the first subcomponent being configured to include
(1) one or more spectral illumination light sources aligned with a first set of conduits of the plurality of conduits, each of the one or more spectral illumination light sources is configured to emit light of a particular central wavelength through a corresponding conduit of the first set of conducts for propagation through the second subcomponent toward the tissue under analysis, and
(2) one or more light detection elements aligned with a second set of conduits of the plurality of conduits, each of the one or more light detection elements is configured to collect the received light propagating through an opening of the second subcomponent and a conduit of the second set of conduits and determine reflectance spectra associated with the received light; and
network interface logic for support communications with a cloud network,
wherein at least one of the handle or the head portion of the diagnostic tool includes analytic components that are configured to (i) generate diagnostic metrics that include characteristics of the reflectance spectra and (ii) compare the characteristics of the reflectance spectra to data associated with characteristics of known reflectance spectra associated with at least one of healthy tissue or tissue of patients exhibiting health conditions to generate results for upload to a cloud network via a network interfaces.

22. The diagnostic tool of claim 21, wherein the first subcomponent of the head portion further includes one or more imaging components aligned with a third set of conduits of the plurality of conduits, each of the one or more imagining components being configured to capture one or more images associated with an ear canal including the tissue of analysis.

23. The diagnostic tool of claim 21, wherein the one or more imaging components are configured to identify (i) an ailment associated with the tissue under analysis or confirm that the tissue is healthy and (ii) capture a degree of distention of an eardrum to detect a bulging or retracting tympanic membrane.
